(19) United States
(12) Patent Application Publication
(10) Pub. No.: US 2024/0307252 A1
(43) Pub. Date: Sep. 19, 2024

(54) MASSAGE APPARATUS AND METHOD OF CONTROLLING THE SAME

(71) Applicant: SAMSUNG ELECTRONICS CO., LTD., Suwon-si (KR)

(72) Inventors: Taeseob LIM, Suwon-si (KR); Daesung KI, Suwon-si (KR)

(73) Assignee: SAMSUNG ELECTRONICS CO., LTD., Suwon-si (KR)

(21) Appl. No.: 18/411,479

(22) Filed: Jan. 12, 2024

Related U.S. Application Data
(63) Continuation of application No. PCT/KR2023/021078, filed on Dec. 20, 2023.

(30) Foreign Application Priority Data
Mar. 15, 2023 (KR) 10-2023-0034157

(51) Int. Cl. A61H 15/00

(52) U.S. Cl. CPC A61H 15/0078; A61H 2201/0149; A61H 2201/5043; A61H 2205/022

(57) ABSTRACT

A massage apparatus includes a seat frame including a head seating portion on which a head of a user is seated so that a face of the user is placed forward, and a body seating portion, a base frame configured to support the seat frame, a massage module coupled to the base frame and configured to control a massage function, a cover rotatably coupled to the base frame, and placeable at a first position or a second position which the cover is rotated from the first position to cover the head seating portion, at least one sensor to obtain a sensor value to estimate a face position of the user, a display to display an image on an inside of the cover at the second position, and a controller configured to adjust a position of the image displayed by the display based on the obtained sensor value.

### FIELD

The disclosure relates to a massage apparatus and a method of controlling the same.

### DESCRIPTION OF THE RELATED ART

In general, massage apparatuses are apparatuses that are provided for a user to receive a massage while seated, providing a massage to an entire body of the user.

A massage apparatus includes a massage module for providing a massage to a user's body therein. The massage module performs a massage by applying appropriate pressure to the user's body through rotational and reciprocating motions.

A massage apparatus may typically be provided as a chair-type massage apparatus or a bed-type massage apparatus. Recently, a massage apparatus has been provided that may be used as a chair-type, but may also be changed to a bed-type depending on the user's needs.

On the other hand, there is a demand for a massage apparatus that allows a user to watch a screen through the massage apparatus while receiving a massage on the massage apparatus or while not receiving a massage on the massage apparatus.

### SUMMARY

The disclosure is directed to providing a massage apparatus that allows a user to watch a screen while receiving a massage or not receiving a massage.

Further, the disclosure is directed to providing a massage apparatus capable of adjusting a position of an image in response to the height and/or eye level of the user.

Technical tasks to be achieved in this document are not limited to the technical tasks mentioned above, and other technical tasks not mentioned will be clearly understood by those skilled in the art from the description below.

An embodiment of the disclosure provides a massage apparatus including a seat frame including a head seating portion on which a head of a user is seated, and a body seating portion on which a body of the user is seated, a base frame configured to support the seat frame, a massage module coupled to the base frame and configured to control a massage function, a cover rotatably coupled to the base frame, and placeable at a first position or a second position which the cover is rotated from the first position to cover the head seating portion, at least one sensor to obtain a sensor value to estimate a face position of the user, a display configured to display an image on the cover based on the cover being at the second position, and a controller configured to adjust a position of the image displayed by the display based on the obtained sensor value.

The controller may be configured to adjust the position of the image displayed by the display based on the sensor value obtained from the at least one sensor during an operation of the massage module.

The at least one sensor may include a pressure sensor to obtain a pressure value so that the controller is configured to determine a sitting height and a face position of the user based on the obtained pressure value.

The at least one sensor may include a camera coupled to the cover, and the controller may be configured to adjust the image displayed by the display based on the image obtained from the camera.

The controller may be configured to determine whether the user is asleep based on the obtained sensor value.

The controller may be configured to adjust brightness of the image displayed by the display based on the image obtained from the camera.

The base frame may include a left side to cover a left side of the face of the user, and a right side to cover a right side of the face of the user, and the massage apparatus may further include a plurality of speakers arranged on the left and right sides of the base frame, respectively, and configured to transmit sound toward the user.

The controller may be configured to adjust a volume of the plurality of speakers based on the image obtained from the camera.

The plurality of speakers may include a left speaker disposed on the left side of the base frame and a right speaker disposed on the right side of the base frame, and the controller may be configured to decrease an output intensity of the left speaker or an output intensity of the right speaker as a left ear of the user or a right ear of the user is closer to the left speaker or the right speaker, respectively, and to increase the output intensity of the left speaker or the right speaker as the user's left ear or right ear is further away from the left speaker or the right speaker.

The cover may include a left side to cover the left side of the base frame and the left side of the face of the user, and a right side to cover the right side of the base frame and the right side of the face of the user.

The display may include a projector disposed on an upper portion of the head seating portion.

The display may be configured to display the image on an inside of the cover based on the cover being at the second position.

An embodiment of the disclosure provides a method of controlling a massage apparatus including a base frame, a massage module coupled to the base frame, a cover rotatably coupled to the base frame, and a display configured to display an image on the cover, the method including covering the base frame by rotating the cover, obtaining a sensor value to estimate a face position of a user via at least one sensor, and adjusting a position of the image displayed by the display based on the obtained sensor value.

The display may be configured to display the image on an inside of the cover.

The at least one sensor may include a sensor to obtain a pressure value detected by the massage module, and the adjusting of the position may include adjusting the position of the image displayed by the display by obtaining the pressure value detected by the massage module during an operation of the massage module.

The at least one sensor may include a camera coupled to the cover, and the adjusting of the position may include adjusting the image displayed by the display based on the image obtained from the camera.

An embodiment of the disclosure provides a method of controlling an massage apparatus according to an embodiment including the base frame, a massage module coupled to the base frame, a cover rotatably coupled to the base frame, a display displaying an image on the cover, and a camera coupled to the cover, the method including adjusting the image displayed by the display based on the image obtained from the camera.

### BRIEF DESCRIPTION OF THE DRAWINGS

FIG. 1 is a perspective view illustrating a massage apparatus according to an embodiment of the disclosure.

FIG. 2 is a perspective view illustrating the massage apparatus according to an embodiment of the disclosure.

FIG. 3 is a perspective view illustrating the massage apparatus according to an embodiment of the disclosure.

FIG. 4 is a perspective view illustrating a massage module in the massage apparatus according to one embodiment of the disclosure.

FIG. 5 is a side cross-sectional view illustrating the massage apparatus according to an embodiment of the disclosure.

FIG. 6 is an enlarged view of the massage apparatus shown in in FIG. 5.

FIG. 7 is a control block diagram showing the massage apparatus according to an embodiment of the disclosure.

FIG. 8 is a control flowchart showing the massage apparatus according to an embodiment of the disclosure.

FIG. 9 is a control flowchart showing the massage apparatus according to an embodiment of the disclosure.

### DETAILED DESCRIPTION

Embodiments described in the disclosure and configurations shown in the drawings are merely examples of the embodiments of the disclosure and may be modified in various different ways at the time of filing of the present application to replace the embodiments and drawings of the disclosure.

In addition, the same reference numerals or signs shown in the drawings of the disclosure indicate elements or components performing substantially the same function.

Also, the terms used herein are used to describe the embodiments and are not intended to limit and/or restrict the disclosure. The singular forms “a,” “an” and “the” are intended to include the plural forms as well, unless the context clearly indicates otherwise. In this disclosure, the terms “including”, “having”, and the like are used to specify features, figures, steps, operations, elements, components, or combinations thereof, but do not preclude the presence or addition of one or more of the features, figures, steps, operations, elements, components, or combinations thereof.

It will be understood that, although the terms first, second, primary, secondary, etc., may be used herein to describe various elements, but elements are not limited by these terms. These terms are only used to distinguish one element from another element. For example, without departing from the scope of the disclosure, a first element may be termed as a second element, and a second element may be termed as a first element. The term of “and/or” includes a plurality of combinations of relevant items or any one item among a plurality of relevant items.

In addition, as used in the disclosure, the meaning of “identical” may include having similar properties or being similar within a certain range. Also, identical may include “substantially the same”. Substantially the same should be understood to include within the scope of “identical” figures that fall within a margin of error in manufacturing, or figures that fall within a margin of difference that is not meaningful with respect to a reference figure.

Furthermore, terms such as “part”, “unit”, “block”, “member”, “module”, and the like may refer to a unit that processes at least one function or operation. For example, these terms may refer to at least one piece of hardware, such as a field-programmable gate array (FPGA)/an application specific integrated circuit (ASIC), at least one piece of software stored in memory, or at least one process processed by a processor.

Meanwhile, in the disclosure, for ease of description, a first direction X may be referred to as a front-back direction, a second direction Y may be referred to as a left-right direction, and a third direction C may be referred to as an up-down direction. However, the terms “front”, “rear”, “left”, “right”, and the like used in the following description are defined based on the drawings, and the shape and location of each component may not be limited by these terms.

Hereinafter, an embodiment according to the disclosure will be described in detail with reference to the accompanying drawings.

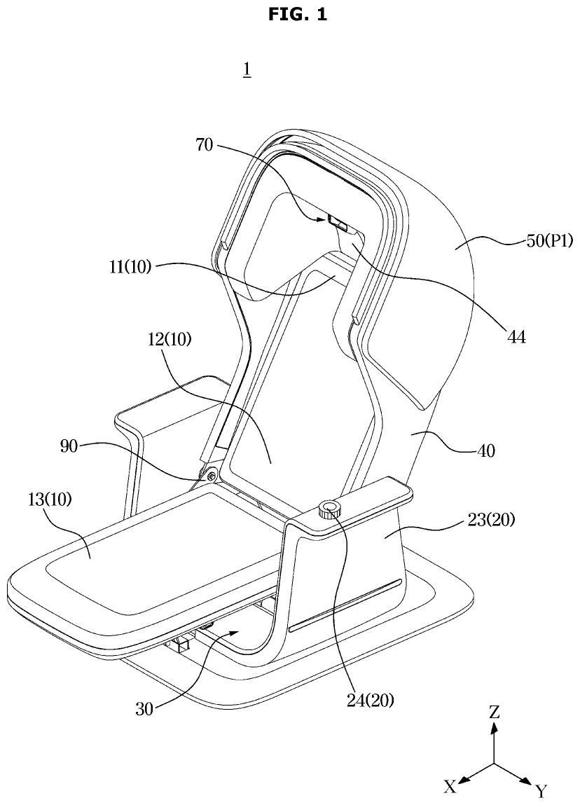

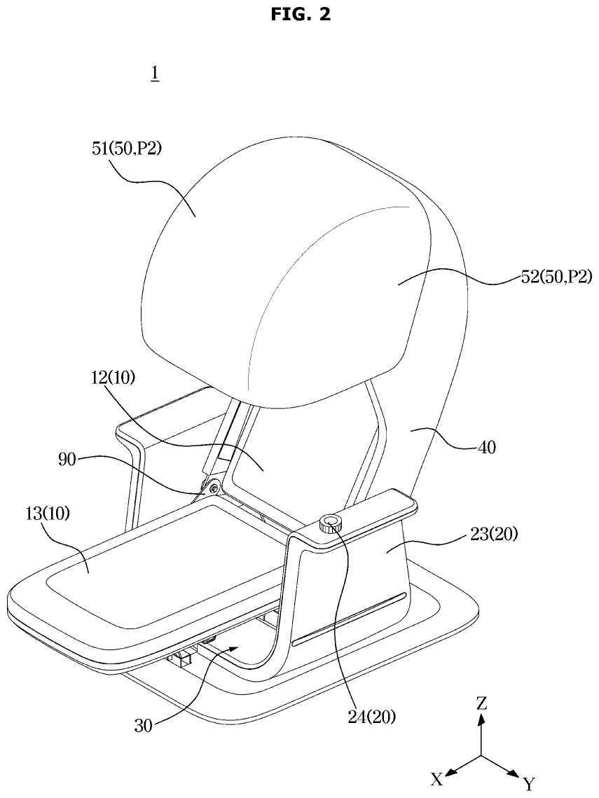

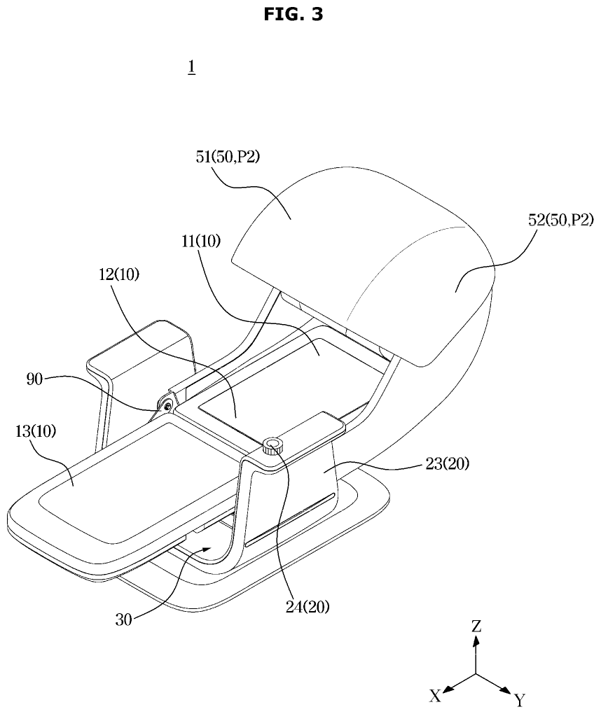

FIG. 1 is a perspective view illustrating a massage apparatus according to an embodiment of the disclosure. FIG. 2 is a perspective view illustrating the massage apparatus according to an embodiment of the disclosure. FIG. 3 is a perspective view illustrating the massage apparatus according to an embodiment of the disclosure.

Referring to FIGS. 1 to 3, a massage apparatus 1 may be an apparatus for providing a massage to a user. The massage apparatus 1 may be implemented in the form of a chair so that the user may relax. The massage apparatus 1 may be covered with a leather or fabric material that may be in contact with the user's skin. As a result, such a soft material may come into contact with the user's skin, so that the user may feel psychologically stable.

In the massage apparatus according to an embodiment of the disclosure, a cover may be rotatable. FIG. 1 is a view when the cover of the massage apparatus does not cover a seating portion, and FIG. 2 is a view when the cover of the massage apparatus covers the seating portion.

FIGS. 1 and 2 show the massage apparatus when used in a chair mode, and FIG. 3 shows the massage apparatus when used in a bed mode. The massage apparatus according to an embodiment may be freely transformed from the chair mode to the bed mode or from the bed mode to a massage type structure.

The massage apparatus according to an embodiment may provide a massage function regardless of whether a cover 50 is open or closed, and regardless of the massage apparatus is in the chair mode or in the bed mode.

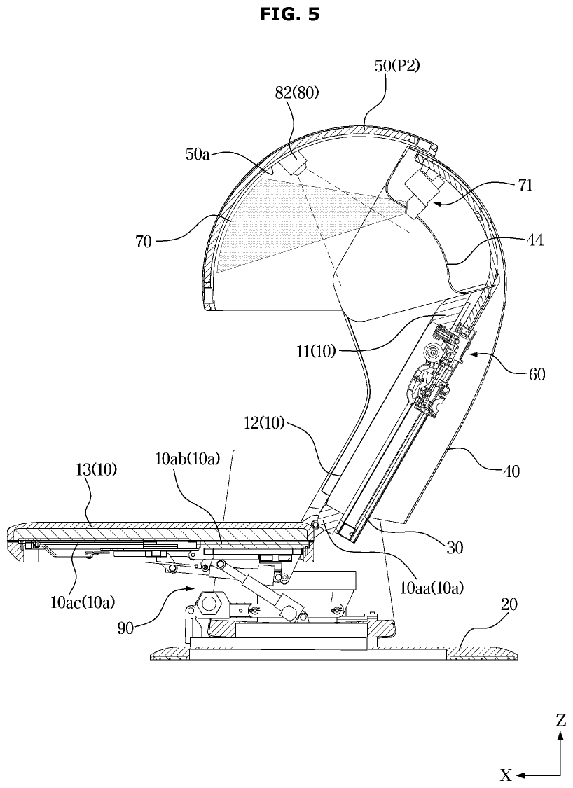

The massage apparatus 1 may include a seat portion 10 for supporting the user. The seat portion 10 may include a seat frame 10a that forms a framework of the seat portion 10 (see FIG. 5). The seat frame 10a may extend so as to support from the user's head to an upper body and a lower body. The seat portion 10 may include a first side and a second side opposite to the first side. For example, while the massage apparatus is in the chair mode, the first side may be a front side and the second side may be a rear side. The first side and the second side of the seat portion 10 may be the first side and the second side of the seat frame. Seating portions 11, 12, and 13 may be arranged on the first side.

The seat frame 10a may include a first seat frame 10aa, a second seat frame 10ab, and a third seat frame 10ac. For example, the first seat frame 10aa may support the upper body and a portion of the buttocks. The second seat frame 10ab may support the lower body, such as the buttocks and thigh sides. The third seat frame 10ac may support the lower body, such as the calf and foot sides (see FIG. 5).

The first seat frame 10aa and the second seat frame 10ab may be rotatably coupled. The first seat frame 10aa and the second seat frame 10ab may be coupled by a rotation support frame 90. The second seat frame 10ab and the third seat frame 10ac may be rotatably coupled. In addition, the second seat frame 10ab and the third seat frame 10ac, which support the lower body of the user, may be freely adjusted and used by the user.

For example, in the chair mode of the massage apparatus, the first seat frame 10aa may be coupled at an upper portion of the second seat frame 10ab and be disposed at a rear side of the second seat frame 10ab. In addition, in the bed mode of the massage apparatus, the first seat frame 10aa may be disposed at the rear side of the second seat frame 10ab.

The first seat frame 10aa may support the first seating portion 11 and the second seating portion 12, and the second seat frame 10ab may support the third seating portion 13. The first seat frame 10aa may be formed integrally with the first seating portion 11 and the second seating portion 12. The second seat frame 10ab may be formed integrally with the third seating portion 13.

The seat portion 10 may include the seating portions 11, 12, and 13. The seating portions 11, 12, and 13 may be provided on one side of the seat portion 10. For example, the seating portions 11, 12, and 13 may be provided on the first side of the seat portion 10. For example, the seat frame 10a may be coupled to a front side of a base frame 30 to allow the user's body to be seated and supported. The seating portions 11, 12, and 13 may be referred to as seating surfaces.

The seating portions 11, 12, and 13 may be provided to contact the user's body. For example, the seating portions 11, 12, and 13 may be in contact with a back of the user's body to support the user from behind.

The seating portions 11, 12, and 13 may include a soft material to increase the seating comfort of the user. In addition, the seating portions 11, 12, and 13 may include a deformable material to allow the back of the user's body to be pressed by a massage module 60 located at a rear side of the seat frame 10a. However, the material forming the seating portions 11, 12, and 13 is not limited thereto, and any material which provides comfort to the user is sufficient.

The upper body of the user and the lower body of the user, as described below, may be distinguished based on the user's buttocks.

The seat portion 10 is not limited to what is shown in the drawings, and may be formed to surround the user's head, back, arms, legs, and buttocks, respectively. The massage module 60 may be provided on one side of the seat portion 10 so as to provide a massage to the user.

The seat portion 10 may include the first seating portion 11, the second seating portion 12, and the third seating portion 13. The first seating portion 11 may support a portion of the user's head, the entire head, and the upper body. The first seating portion may be referred to as the head seating portion 11.

The second seating portion 12 may be coupled to one end of the first seating portion 11. For example, while the massage apparatus is used in the chair mode, the second seating portion 12 may be connected to the first seating portion 11 and thus positioned below the first seating portion 11. The second seating portion 12 may be provided to support a portion of a rear side of the buttocks of the lower half of the user's body.

The third seating portion 13 may be coupled to one end of the second seating portion 12. For example, while the massage apparatus is used in the chair mode, the third seating portion 13 may be connected to the second seating portion 12 and thus positioned below the second seating portion 12. The third seating portion 13 may be provided to support a portion of the lower half of the user's body or the entire lower half.

For example, the first seating portion 11 and the second seating portion 12 may support a portion or the entire upper half of the user's body, including the user's head, from a rear side of the user. The third seating portion 13 may support a portion or the entire lower half of the user's body from the rear side of the user. The second seating portion 12 and the third seating portion 13 may be referred to as a body seating portion.

The second seating portion 12 may be rotatably coupled to the third seating portion 13 to allow an angle at which the second seating portion 12 supports the user's upper body to be adjusted. For example, the second seating portion 12 may be provided to be rotatable in a front-back direction with respect to the third seating portion 13 based on a point where the second seating portion 12 is connected to the third seating portion 13.

However, the disclosure is not limited thereto, and the second seating portion 12 may be fixed to the third seating portion 13.

The third seating portion 13 may be provided to support the user's thighs and calves. For example, while the massage apparatus is used in the chair mode, the third seating portion 13 may support the user from the rear side of the user.

However, although not shown in the drawings, a portion supporting the user's calf in the third seating portion 13 may be provided in a shape capable of surrounding the user's calf so as to accommodate the user's calf.

The massage apparatus 1 may include the rotation support frame 90. The rotation support frame 90 may allow the seat frame to be rotated.

The massage apparatus 1 may include a leg 20 provided to support the seat portion 10 and the base frame 30. The leg 20 may be coupled below the seat frame 10a and the base frame 30.

The leg 20 may be provided on the left, on the right, and under the seat frame 10a and the base frame 30. The leg 20 is not limited to that shown in the drawings and may be provided as at least one or more legs, and may be formed as a separate element on the base frame 30.

The massage apparatus may include a user interface 24. The user interface 24 may be provided on an armrest portion 23. The user interface 24 may be manipulated by the user to operate the massage apparatus in a massage-on mode or a massage-stop mode. Furthermore, the user may manipulate the user interface 24 to cause the shape of the massage apparatus to be in the chair mode or in the bed mode. The user interface 24 may be referred to as an operating device 24. The user interface 24 may be provided to be rotatable, and the user may rotate the user interface 24 to input instructions to the massage apparatus. The user interface 24 may include an input interface and an output interface, respectively.

The base frame 30 may be located on one side of the seat frame 10a. For example, while the massage apparatus is used in the chair mode, the base frame 30 may be positioned below and behind the seat frame 10a. In addition, while the massage apparatus is used in the bed mode, the base frame may be positioned below the seat frame 10a.

The massage apparatus may include a rear frame 40. While the massage apparatus is used in the chair mode, the rear frame 40 may be disposed on the rear side of the seat frame 10a and the base frame 30. In addition, while the massage apparatus is used in the bed mode, the rear frame 40 may be disposed below the seat frame 10a and the base frame 30.

The rear frame 40 may be provided to surround the seat frame 10a and the base frame 30. The rear frame 40 may surround the user while the user is using the massage apparatus. The rear frame 40 may be referred to as the rear cover 40.

An accommodation space (not shown) may be formed within the rear frame 40. When the rear frame 40 is coupled with the seat portion 10 and the base frame 30, the accommodation space (not shown) may accommodate at least the user's head and/or a portion of the upper half of the user's body. In addition, the base frame 30 may be accommodated in the accommodation space (not shown).

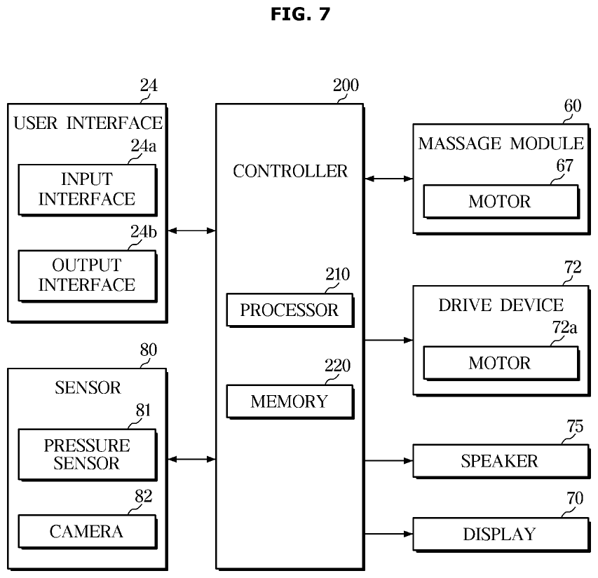

A speaker 75 (see FIG. 7) may be accommodated in the rear frame 40. The speaker 75 may be placed on both sides of the rear frame 40 to cover both ears of the user. Because the rear frame 40 may be included in the base frame 30, the speakers 75 may be disposed on both sides of the base frame 30.

The rear frame 40 may include a seating portion 44. The seating portion 44 of the rear frame 40 may be a head seating portion 44 for supporting a back side of the user's head. The seating portion 44 of the rear frame 40 may be covered by the cover while the cover is in a second position. The head seating portion 44 and the first seating portion 11 may be connected.

The rear frame 40 and the leg 20 are described as a separate configuration from the base frame 30, but the rear frame 40 and the leg 20 may also be included in the base frame 30.

The seat frame 10a, the base frame 30, and the rear frame 30 described above are only examples, and the seat frame 10a, the base frame 30, and the rear frame 30 may be provided in various shapes as long as they support the user's body.

The massage apparatus may include the cover 50. The cover 50 may be rotatably coupled to the rear frame 40. The cover 50 may be rotated by the user by manipulating the user interface 24 as needed. For example, the cover 50 may have a first position P1, and a second position P2 that is rotated from the first position P1 to cover the first seating portion 11.

The cover 50 may include a first cover portion 51 and a second cover portion 52. The first cover portion 51 may have a certain curvature and form an upper portion of the cover 50. The second cover portion 52 may be provided on the left and right sides of the first cover portion 51. The second cover portion 52 may be coupled to the rear frame 40 to cover both sides of the user. The second portion 52 may be left and right sides of the cover 50.

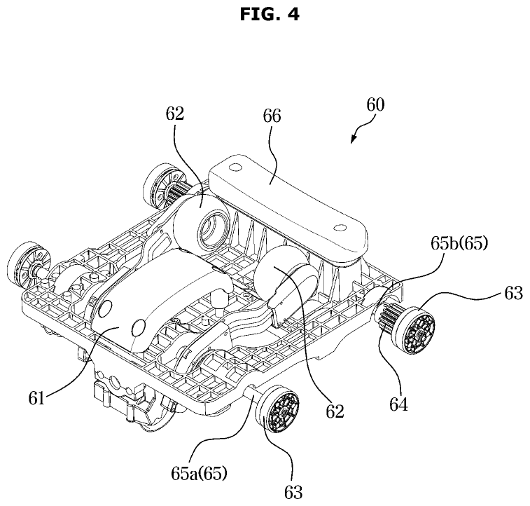

FIG. 4 is a perspective view illustrating the massage module in the massage apparatus according to an embodiment of the disclosure.

Referring to FIG. 4, the massage apparatus may include the massage module 60. The massage module 60 may be disposed on the second side of the seat frame 10a. The massage module 60 may provide a massage to the user. For example, while the massage apparatus is used in the chair mode, the massage module 60 may be positioned at the rear side of the first seating portion 11 and the second seating portion 12 to provide a massage from the head to the waist of the user. In addition, while the massage apparatus is used in the bed mode, the massage module 60 may be positioned below the first seating portion 11 and the second seating portion 12 to provide a massage from the head to the waist of the user.

Furthermore, the disclosure is not limited thereto, and a separate module capable of providing a massage partially or fully to the lower body of the user may be provided on the seat frame 10a separately from the massage module 60 shown in the drawings.

The massage module 60 may provide a massage to the user. The massage module 60 may include a motor, a massage roller 62, a moving roller 63, a pinion gear 64, and a drive shaft 65.

The motor may be connected to the drive shaft 65 to transmit power to the massage roller 62 and the moving roller 63. For example, the motor may be connected to a first drive shaft 65a to transmit power to the massage roller 62, thereby allowing the massage module 60 to provide the massage function to the user. In addition, the motor may be connected to the first drive shaft 65a and a second drive shaft 65b to transmit power to the moving roller 63, so that the massage module 60 may be moved within an opening, which is formed in the base frame 30, along a guide rack gear (not shown).

The massage roller 62 may be driven by the motor and the first drive shaft 65a to provide a massage (e.g., a tapping function) to the user. The massage roller 62 may be provided in a plurality.

The moving roller 63 may receive power from the motor, the first drive shaft 65a, and the second drive shaft 65b to allow the massage module 60 to move within the opening formed in the base frame 30. The moving roller 63 may be disposed within a guide receiving space formed in the base frame 30. The moving roller 63 may be provided in a plurality. For example, four moving rollers 63 may be provided. However, the number of moving rollers 63 is not limited to the above examples.

The pinion gear 64 may mesh with the guide rack gear to guide the movement of the massage module 60. The pinion gear 64 may be disposed within the guide receiving space. The pinion gear 64 may be formed to correspond to a rack gear of the base frame 30. The pinion gear 64 may be provided in a plurality.

The drive shaft 65 may transmit power from the motor to the massage rollers 62 and the moving rollers 63. The drive shaft 65 may extend along the left-right direction. The drive shaft 65 may include the first drive shaft 65a and the second drive shaft 65b. The first drive shaft 65a may be connected to the massage rollers 62 and the moving rollers 63. The second drive shaft 65b may be connected to the moving rollers 63 and the pinion gear 64.

The massage module 60 may further include supporters 61 and 66. The supporters 61 and 66 may support the user during the massage-on mode after the user is comfortably reclined or leaned against the massage apparatus. The supporters 61 and 66 may include the first supporter 61 and a second supporter 66. While the massage apparatus is in the chair mode, the first supporter 61 may be placed below the second supporter 66. At this time, the first supporter 61 may extend in a vertical direction, and the second supporter 66 may extend in a lateral direction (i.e., the left-right direction). The first supporter 61 and the second supporter 66 may be spaced apart from each other.

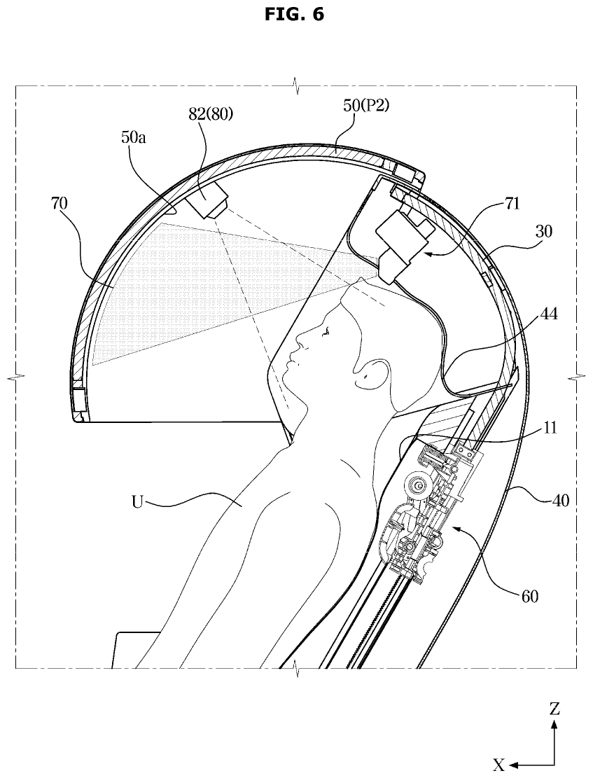

FIG. 5 is a side cross-sectional view illustrating the massage apparatus according to an embodiment of the disclosure. FIG. 6 is an enlarged view of the massage apparatus shown in FIG. 5.

Referring to FIGS. 5 and 6, the massage apparatus according to an embodiment may further include a display 70.

The display 70 may display an image (including a video). For example, the display 70 may display the image on an inner side 50a of the cover 50 at the second position P2 of the cover 50 to allow a user U to watch the image while using the massage apparatus. The inner side 50a of the cover may refer to a side facing the user's face and the seating portions 11, 12, and 13.

The display 70 may include a projector 71. For example, the projector 71 may be the beam projector 71. The projector 71 may emit a beam toward the inner side 50a of the cover 50 to display the image on the inner side 50a of the cover 50. The projector 71 may be placed above the head seating portion 11. In addition, the projector 71 may be placed above the head seating portion 44. The projector 71 may adjust a position of the image by a controller, which will be described later.

Furthermore, the display 70 may include a curved display disposed on the inner side 50a of the cover. For example, the image displayed on the inner side 50a of the cover may be displayed by the curved display 70. The curved display may adjust the position of the image by a controller to be described later. However, examples of displays are not limited to the above examples.

The massage apparatus according to an embodiment may further include a sensor 80. At least one sensor 80 may be provided. The sensor 80 may obtain a sensor value for estimating a location of the users face.

The at least one sensor 80 may include a camera 82. The camera 82 may estimate the location of the users face. The camera 82 may be placed on the inner side 50a of the cover 50. For example, the camera 82 may be placed above the face to estimate an eye level of the user. However, a position of the camera 82 is not limited to the above example. The camera 82 may be referred to as a face detection sensor.

FIG. 7 is a control block diagram of the massage apparatus according to an embodiment of the disclosure.

Referring to FIG. 7, in an embodiment, the massage apparatus 1 may include a user interface 24, the massage module 60, a drive device 72, the speaker 75, the display 70, the sensor 80, and a controller 200.

The user interface 24 may provide a user interface for interaction between the user and the massage apparatus 1.

The user interface 24 may include at least one input interface 24a and at least one output interface 24b.

The at least one input interface 24a may convert sensory information received from the user into an electrical signal.

The at least one input interface 24a may include a power button, an operation button, a course selection dial (or course selection button), and a massage-on mode/massage-stop mode/chair mode/bed mode setting button. The at least one input interface 24a may include, for example, a tact switch, a push switch, a slide switch, a toggle switch, a micro switch, a touch switch, a touch pad, a touch screen, a jog dial, and/or a microphone.

The at least one output interface 24b may communicate various data related to the operation of the massage apparatus 1 to the user by generating the sensory information.

For example, the at least one output interface 24b may communicate to the user information related to the massage course and the duration of operation of the massage apparatus 1, the setting of the massage-on mode/massage-stop mode/chair mode/bed mode. The information regarding the operation of the massage apparatus 1 may be output on a screen, indicator, voice, or the like. The at least one output interface 24b may include, for example, a liquid crystal display (LCD) panel, a light emitting diode (LED) panel, a speaker, or the like.

The drive device 72 may include a motor 72a for providing a driving force to the display 70 so as to adjust the position of the image displayed by the display 70. The drive device 72 may operate based on a control signal from the controller 200. Furthermore, the drive device 72 may provide a driving force to rotate the seat frame 10a from the chair mode to the bed mode or from the bed mode to the chair mode. Here, the drive device for providing the driving force to the seat frame 10a may be provided separately.

The massage module 60 may include the massage roller 62, the moving roller 63, the pinion gear 64, and a motor 67 that transmits a driving force to the drive shaft 65. The motor 67 may be operated based on a control signal from the controller 200.

The speaker 75 may output sound for the image displayed on the display. Alternatively, the speaker 75 may provide sound to the user regardless of the presence or absence of the image. The speaker 75 may be placed on the base frame 30 to transmit sound towards the user.

A plurality of speakers 75 may be provided, and the plurality of speakers 75 may include a left speaker disposed on the users left side and a right speaker disposed on the users right side. The plurality of speakers 75 may adjust the output intensity from each speaker 75 depending on the position of the user's face and ears.

The sensor 80 may include at least one sensor obtaining information related to a state of the user U. For example, the sensor 80 may obtain the sensor value for estimating the location of the users face.

For example, the sensor 80 may include at least one of a pressure sensor 81 for detecting pressure applied to the massage module 60 and the camera 82 for recognizing the users face.

The pressure sensor 81 may be connected to the massage module 60 to detect the pressure being applied to the massage module 60 based on the users body area and transmit information regarding the pressure to the controller 200. The controller 200 may determine a position of the massage module 60 and a face position of the user U based on information about the pressure.

For example, the controller 200 may determine, based on a lowered sensor value detected by the pressure sensor 81, that the massage of the users shoulder area is completed and the massage module is located in the neck area. Accordingly, the controller 200 may determine the face and eye level position of the user U.

The controller 200 may control various components of the massage apparatus 1 (e.g., the massage module 60, the drive device 72, the speaker 75, and the display 70). The controller 200 may control various components of the massage apparatus 1 to perform at least one mode including the massage-on mode, the massage-stop mode, the chair mode, the bed mode, and the like, based on user input.

For example, the controller 200 may control the motor 67 to adjust a moving speed of the massage module 60 during the massage mode, or move the massage module 60 to a first end 31a of an opening 31 in response to the massage mode being switched to the massage-stop mode.

Alternatively, the controller 200 may estimate the face position of the user based on the sensor value and adjust the position of the image displayed by the display 70 based on the estimated face position.

In addition, the controller 200 may adjust the brightness of the image displayed by the display 70 based on the image obtained from the camera 82. For example, upon determining that the user is asleep by the camera 82, which is one of the sensors 80, the controller 200 may dim the brightness of the image. Conversely, upon determining that the user has awakened from sleep, the controller 200 may brighten the image again.

In addition, the controller 200 may control the plurality of speakers 75. For example, the controller 200 may decrease the output intensity of the left speaker as the users left ear is closer to the left speaker. In addition, for example, the controller 200 may increase the output intensity of the left speaker as the users left ear is further away from the left speaker. Conversely, the controller 200 may also control the plurality of speakers 75 when the right ear is near or far from the speaker.

In addition, for example, upon determining that the user is asleep by the camera 82, which is one of the sensors 80, the controller 200 may decrease the output of the speakers 75. Conversely, upon determining that the user has awakened from asleep, the controller 200 may increase the output of the speakers 75.

The controller 200 may include hardware, such as a central processing unit (CPU), a micro-computer (Micom), or memory, and software, such as a control program. For example, the controller 200 may include at least on memory 220 storing data in the form of algorithms and/or programs for controlling the operation of components in the massage apparatus 1, and at least one processor 210 performing the operation described above and the operation to be described later using data stored in the at least one memory 220. The memory 220 and the processor 210 may each be implemented as a separate chip. The processor 210 may include one or two or more processor chips, or may include one or two or more processing cores. The memory 220 may include one or two or more memory chips, or may include one or two or more memory blocks. Additionally, the memory 220 and processor 210 may be implemented on a single chip.

The massage apparatus may include a temperature control device (not shown). The temperature control device may control the temperature based on the input interface or may adjust the temperature based on whether the user is sleeping or not.

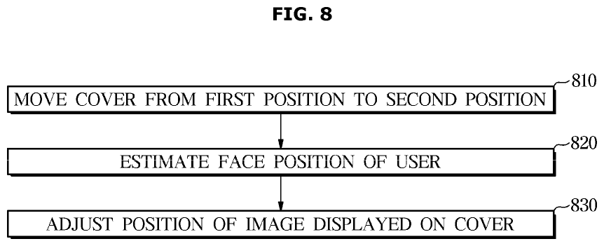

FIG. 8 is a control flowchart illustrating the massage apparatus according to an embodiment of the disclosure.

Referring to FIG. 8, an exemplary process of a method by which the massage apparatus 1 according to an embodiment may adjust the position of the image by estimating the face position of the user U is shown.

In the massage apparatus according to an embodiment, the cover 50 may be moved from the first position P1 to the second position P2 (operation 810).

The controller 200 may allow the cover 50 to move from the first position P1, which opens the head seating portion 11, to the second position P2, which covers the head seating portion 11, based on user input. If it is bright daylight or a light is on in the room, the cover 50 may move to the second position P2 to provide a dark environment for the user U.

Furthermore, the cover 50 may cover both ears of the user U, thus reducing the noise outside the massage apparatus.

The controller 200 may estimate the face position of the user U based on the sense value obtained by the at least one sensor 80 (operation 820).

For example, the pressure sensor 81 connected to or disposed on the massage module 60 may transmit a changing pressure value to the controller 200. The controller 200 may determine the sitting height and face position of the user U based on a section where the pressure value decreases.

Alternatively, for example, the camera 82 may transmit an image value to the controller 200. The controller 200 may determine the sitting height and face position of the user U based on information received from the camera 82.

In addition, the user U may input a value related to the user's body via the input interface 24a, and the controller 200 may determine the sitting height and face position of the user U based on the value related to the user's body.

In addition, for example, the controller 200 may determine the face position of the user U in consideration of a correlation between the sensor value obtained by the pressure sensor 81 and the value obtained by the camera 82.

The method by which the sensor 80 and the user interface 24 collect the sensor values regarding the face position of the user U, and the method by which the controller 200 determines the face position of the user U are limited to the above examples.

The controller 200 may adjust the position of the image based on the determined face position of the user U (operation 830).

For example, upon determining that the face position of the user U is higher than a reference value, the controller 200 may cause the image displayed on the inner side 50a of the cover 50 to move upward. For example, the controller 200 may actuate the drive device that drives the projector 71 to cause the projector 71 to emit a beam upward. Conversely, for example, upon determining that the face position of the user U is lower than the reference value, the controller 200 may actuate the drive device that drives the projector 71 to cause the projector 71 to emit a beam downward.

Furthermore, for example, the controller 200 may adjust the position of the image based on the face position of the user U determined by the user's input.

Furthermore, for example, the controller 200 may adjust an image display position of the curved display 70 by operating the drive device that drives the curved display 70.

According to an embodiment, the controller 200 may adjust the height of the image by estimating the face and/or eye height of the user U regardless of the user's input, thereby increasing the convenience of the user U of the massage apparatus. In addition, the user U of the massage apparatus according to an embodiment may be able to watch the image with greater immersion due to the increased convenience.

According to an embodiment, the massage apparatus may provide the image to fit the eye level of the user U regardless of the sitting height of the user U. Accordingly, the user U may comfortably watch the image on the massage apparatus without having to excessively bend the neck or place a support on the buttocks. The massage apparatus may provide the image tailored to the eye level of the user U regardless of the height at which the user U is seated, thereby preventing the user U from having a herniated cervical disc and/or poor posture.

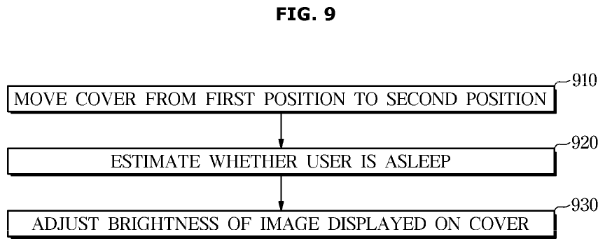

FIG. 9 is a control flowchart illustrating the massage apparatus according to an embodiment of the disclosure.

Referring to FIG. 9, an exemplary process by which the massage apparatus 1 according to an embodiment may adjust the brightness and/or volume of the image by estimating whether the user U is asleep is shown.

In the massage apparatus according to an embodiment, the cover 50 may be moved from the first position P1 to the second position P2 (operation 910).

The controller 200 may allow the cover 50 to move from the first position P1, which opens the head seating portion 11, to the second position P2, which covers the head seating portion 11, based on the user input. If it is bright daylight or a light is on in the room, the cover 50 may move to the second position P2 to provide a dark environment for the user U.

Furthermore, the cover 50 may cover both ears of the user U, thus reducing the noise outside the massage apparatus.

The controller 200 may estimate whether the user U is asleep based on the value obtained by the at least one sensor 80 (operation 920).

For example, the camera 82 may transmit the image value to the controller 200. The controller 200 may determine whether the user U is asleep based on the information received from the camera 82. For example, if the user U closes his or her eyes or does not move for a certain period of time, the controller 200 may estimate that the user U has entered a sleep state.

Furthermore, for example, the user U may cause the massage apparatus to enter a sleep mode after a certain period of time via the input interface 24a.

The method by which the sensor 80 and the user interface 24 collect information about whether the user U is asleep, and the method by which the controller 200 determines whether the user U is asleep are not limited to the above examples.

The controller 200 may adjust the brightness of the image based on whether the user U is asleep (operation 930). Furthermore, the controller 200 may adjust the volume of the image based on whether the user U is asleep.

For example, the controller 200 may estimate that the user U has entered the sleep state when the user U closes his or her eyes or does not move for a certain period of time. Upon determining that the user U has entered the sleep state, the controller 200 may lower the brightness of the image or decrease the volume.

For example, the controller 200 may cause the projector 71 to lower the brightness of the image. Conversely, for example, upon determining that the user U has awakened, the controller 200 may cause the projector 71 to increase the brightness of the image.

Furthermore, for example, upon determining that the user U is asleep, the controller 200 may reduce the output of the speakers 75. Conversely, upon determining that the user U has awakened from sleep, the controller 200 may increase the output of the speakers 75.

According to an embodiment, the massage apparatus may determine whether the user U is asleep, thereby minimizing disturbance while the user U is asleep.

According to one aspect of the present disclosure, the massage apparatus capable of watching the image may be provided via the display 70 that displays the image on the inner side 50a of the cover 50.

Further, according to one aspect of the present disclosure, the massage apparatus may be provided that may adjust the position of the image by estimating the face position of the user U through the sensor 80.

The effect of one aspect of the present disclosure is not limited to the effects mentioned above, and other effects not mentioned may be clearly understood by those skilled in the art from the following description.

The massage apparatus according to an embodiment may include the seat frame 10a including the head seating portion 11 on which the face of the user U is seated and the body seating portions 12 and 13 on which the body of the user U is seated, the base frame 30 configured to support the seat frame 10a, the massage module coupled to the base frame 30 and configured to provide the massage function, the cover 50 rotatably coupled to the base frame 30 and placeable at the first position P1 and the second position P2 that is rotated from the first position P1 to cover the head seating portion 11, at least one sensor 80 obtaining the sensor value for estimating the face position of the user U, the display 70 configured to display the image on the cover 50 at the second position P2, and the controller 200 configured to adjust the position of the image displayed by the display 70 based on the sensor value.

In the massage apparatus according to an embodiment, the controller 200 may adjust the position of the image displayed by the display 70 based on the sensor value obtained from the at least one sensor 80 during the operation of the massage module.

In the massage apparatus according to an embodiment, the at least one sensor 80 may include the pressure sensor 81 for obtaining the pressure value detected by the massage module.

In the massage apparatus according to an embodiment, the at least one sensor 80 may include the camera 82 coupled to the cover 50, and the controller 200 may adjust the image displayed by the display 70 based on the image obtained from the camera 82.

In the massage apparatus according to an embodiment, the controller 200 may determine whether the user U is asleep based on the sensor value.

In the massage apparatus according to an embodiment, the controller 200 may adjust the brightness of the image displayed by the display 70 based on the image obtained from the camera 82.

In the massage apparatus according to an embodiment, the base frame 30 may include the left side provided to cover the left face of the user U, and the right side provided to cover the right face of the user U, and may further include the speakers 75 arranged on the left and right sides of the base frame 30 and configured to transmit sound toward the user U.

In the massage apparatus according to an embodiment, the controller 200 may adjust the volume of the speakers 75 based on the image obtained from the camera 82.

In the massage apparatus according to an embodiment, the speakers 75 may include the left speaker disposed on the left side of the base frame 30 and the right speaker disposed on the right side of the base frame 30.

In the massage apparatus according to an embodiment, the controller 200 may decrease the output intensity of the left speaker or the right speaker as the user's left ear or right ear is closer to the left speaker or the right speaker, and to increase the output intensity of the left speaker or the right speaker as the user's left ear or right ear is further away from the left speaker or the right speaker.

In the massage apparatus according to an embodiment, the cover 50 may include the left side provided to cover the left side of the base frame 30 and the left face of the user U, and the right side provided to cover the right side of the base frame 30 and the right face of the user U.

In the massage apparatus according to an embodiment, the display 70 may include the projector 71 disposed on an upper portion of the head seating portion 11.

In the method of controlling the massage apparatus according to an embodiment including the base frame 30, the massage module coupled to the base frame 30, the cover 50 rotatably coupled to the base frame 30, and the display 70 configured to display the image on the cover 50, the method may include covering the base frame 30 by rotating the cover 50, obtaining the sensor value for estimating the face position of the user U via the at least one sensor 80, and adjusting the position of the image displayed by the display 70 based on the sensor value.

In the method of controlling the massage apparatus according to an embodiment, the at least one sensor 80 may include the sensor 81 for obtaining the pressure value detected by the massage module, and the adjusting of the position includes adjusting the position of the image displayed by the display by obtaining the pressure value detected by the massage module during an operation of the massage module.

In the method of controlling the massage apparatus according to an embodiment, the at least one sensor 80 may include the camera 82 coupled to the cover 50, and the adjusting of the position includes adjusting the image displayed by the display 70 based on the image obtained from the camera 82.

In the method of controlling the massage apparatus according to an embodiment including the base frame 30, the massage module coupled to the base frame 30, the cover 50 rotatably coupled to the base frame 30, the display 70 displaying the image on the cover 50, and the camera 82 coupled to the cover 50, the image displayed by the display may be adjusted based on the image obtained from the camera 82.

The method may further include determining whether the user U is asleep based on the image obtained from the camera 82.

While the present disclosure has been particularly described with reference to exemplary embodiments, it should be understood by those of skilled in the art that various changes in form and details may be made without departing from the spirit and scope of the present disclosure.

What is claimed is:

1. A massage apparatus, comprising:
a seat frame including:
a head seating portion on which a head of a user is seated; and
a body seating portion on which a body of the user is seated;
a base frame configured to support the seat frame;
a massage module coupled to the base frame, and configured to control a massage function;
a cover rotatably coupled to the base frame, and placeable at a first position, or a second position which the cover is rotated from the first position to cover the head seating portion;
at least one sensor to obtain a sensor value to estimate a face position of the user,
a display configured to display an image on the cover based on the cover being at the second position; and
a controller configured to adjust a position of the image displayed by the display based on the obtained sensor value.

2. The massage apparatus of claim 1, wherein the controller is configured to adjust the position of the image displayed by the display based on the sensor value obtained from the at least one sensor during an operation of the massage module.

3. The massage apparatus of claim 2, wherein the at least one sensor includes a pressure sensor to obtain a pressure value so that the controller is configured to determine a sitting height and a face position of the user based on the obtained pressure value.

4. The massage apparatus of claim 1, wherein the at least one sensor includes a camera coupled to the cover, and
the controller is configured to adjust the image displayed by the display based on the image obtained from the camera.

5. The massage apparatus of claim 3, wherein the at least one sensor includes a camera coupled to the cover, and
the controller is configured to adjust the image displayed by the display based on the image obtained from the camera.

6. The massage apparatus of claim 4, wherein the controller is configured to determine whether the user is asleep based on the obtained sensor value.

7. The massage apparatus of claim 6, wherein the controller is configured to adjust brightness of the image displayed by the display based on the image obtained from the camera.

8. The massage apparatus of claim 7, wherein
the base frame includes a left side to cover a left side of the face of the user, and a right side to cover a right side of the face of the user, and
the base frame further includes a plurality of speakers arranged on the left and right sides of the base frame, respectively, and configured to transmit sound toward the user.

9. The massage apparatus of claim 8, wherein the controller is configured to adjust a volume of the plurality of speakers based on the image obtained from the camera.

10. The massage apparatus of claim 9, wherein the plurality of speakers includes a left speaker disposed on the left side of the base frame and a right speaker disposed on the right side of the base frame, and
the controller is configured to decrease an output intensity of the left speaker or an output intensity of the right speaker as a left ear of the user or a right ear of the user is closer to the left speaker or the right speaker, respectively, and to increase the output intensity of the left speaker or the right speaker as the user's left ear or right ear is further away from the left speaker or the right speaker.

11. The massage apparatus of claim 9, wherein the cover includes a left side to cover the left side of the base frame and the left side of the face of the user, and a right side to cover the right side of the base frame and the right side of the face of the user.

12. The massage apparatus of claim 1, wherein the display includes a projector disposed on an upper portion of the head seating portion.

13. The massage apparatus of claim 6, wherein the display includes a projector disposed on an upper portion of the head seating portion.

14. The massage apparatus of claim 1, wherein the display is configured to display the image on an inside of the cover based on the cover being at the second position.

15. A method of controlling a massage apparatus including a base frame, a massage module coupled to the base frame, a cover rotatably coupled to the base frame, and a display configured to display an image on the cover, the method comprising:
covering the base frame by rotating the cover,
obtaining a sensor value to estimate a face position of a user through at least one sensor, and
adjusting a position of the image displayed by the display based on the obtained sensor value.

16. The method of claim 15, wherein the display is configured to display the image on an inside of the cover.

17. The method of claim 15, wherein the at least one sensor includes a sensor to obtain a pressure value detected by the massage module, and
the adjusting of the position includes adjusting the position of the image displayed by the display by obtaining the pressure value detected by the massage module during an operation of the massage module.

18. The method of claim 17, wherein the at least one sensor includes a camera coupled to the cover, and
the adjusting of the position includes adjusting the image displayed by the display based on the image obtained from the camera.
